(19) United States
(12) Patent Application Publication
(10) Pub. No.: US 2025/0388868 A1
(43) Pub. Date: Dec. 25, 2025

(54) LEVERAGING TYPE 2 CYTOKINES TO ENHANCE CELL-BASED THERAPY FOR PERIPHERAL ARTERIAL DISEASES

(71) Applicant: The Chinese University of Hong Kong, Hong Kong (CN)

(72) Inventors: Wing Tak, Jack WONG, Hong Kong (CN); Huixian LI, Hong Kong (CN); Chufeng HE, Hong Kong (CN); Yiu Ming CHEUNG, Hong Kong (CN)

(21) Appl. No.: 19/240,284

(22) Filed: Jun. 17, 2025

Related U.S. Application Data
(60) Provisional application No. 63/661,755, filed on Jun. 19, 2024.

(51) Int. Cl. C12N 5/071; A61K 9/00; A61K 35/44; A61P 9/10; C12N 5/077

(52) U.S. Cl. CPC C12N 5/069; A61K 9/0019; A61K 35/44; A61P 9/10; C12N 5/0656; C12N 2501/2304; C12N 2501/2313; C12N 2506/45

(57) ABSTRACT

The subject invention pertains to a novel method for treating Peripheral Arterial Disease (PAD) by leveraging the angiogenic potential of type 2 cytokines IL-4 and IL-13 to enhance the efficacy of induced pluripotent stem cell-derived endothelial cells (iPSC-ECs) and induced endothelial cells derived from fibroblasts (iECs). This present invention aims at enhancing the muscle regeneration and revascularization for obese and diabetes individuals.

### CROSS-REFERENCE TO RELATED APPLICATION

This application claims the benefit of U.S. Provisional Application Ser. No. 63/661,755 filed Jun. 19, 2024, which is hereby incorporated by reference in its entirety including any tables, figures, or drawings.

### BACKGROUND OF THE INVENTION

A Global Burden of Disease Study published in 2023 found that the total number of patients affected by peripheral arterial disease (PAD) almost doubled from 65.8 million in 1990 to 113 million in 2019, with a total number of incidence cases increasing from 6.13 million in 1990 to 11 million in 2019 [1]. Combining aging population and unhealthy lifestyle, the number of people affected by PAD is expected to surpass 200 million within the next decade. Besides, the aging population exceeds the supply of the healthcare system which further prolongs the waiting time for surgical operations.

The progression of PAD will often result in chronic limb-threatening ischemia that is associated with severe complications or even premature death. Particularly, following foot gangrene and ulceration, amputation is an irreversible consequence which is hardly controlled by medical treatment. Surgical intervention is a first-line option for the advanced form of PAD, but not suitable for all PAD patients.

Currently, the pharmacotherapy against PAD is limited. Most of the medical treatments applied in PAD patients are aimed at lowering the comorbidity of major adverse cardiovascular events, including the use of antidiabetic drugs and statin. In fact, another newly emerged drug, cilostazol, has been considered as an option in stimulating angiogenesis and arteriogenesis through upregulating NO production. However, concerns have been raised regarding its side effects, particularly its cardiotoxicity and withdrawal syndrome. Another therapeutic approach involves utilizing the granulocyte-macrophage colony stimulating factor (GM-CSF) to activate the local progenitor cells at the injured sites. However, these cells encounter ischemic stress, which may result in aberrant cell fate.

Existing clinical trials in cell therapy rely on the transplantation of mesenchymal stem cells (e.g., adipose-derived stem cells) or bone marrow mononucleated cells. However, the outcomes are various among different studies. One notable advantage of these cells is their sourcing from a variety of tissues. Nonetheless, while the injection of these stem cells shows promise, these clinical trials lack comprehensiveness in term of sample size and duration. The direct transplantation of stem cells also introduces uncertainty and inconclusive efficacy, carrying classical and undetermined risk factors and potential health concern. In particular, the multipotency of stem cells may lead to the formation of undesired cell types or even clonal hematopoiesis under injury stress, which may compromise the therapeutic efficacy. Therefore, there is a need to develop a safe and effective enhanced cell-based treatment for patients with PAD.

### BRIEF SUMMARY OF THE INVENTION

The subject invention pertains to a method for the treatment of peripheral arterial disease (PAD), utilizing fibroblasts and pluripotent stem cells (iPSCs) collected from a healthy subject to obtain pluripotent stem cells-derived endothelial cells (iPSC-ECs) and induced endothelial cells (iECs), where the iPSC-ECs and iECs are further induced with IL-4 and IL-13 to obtain enhanced

ECs, and where the enhanced ECs are administered to a PAD patient to promote angiogenesis and muscle regeneration in ischemic muscle tissue. In preferred embodiments, the subject is a patient. In more preferred embodiments, the patient is healthy. In embodiments, the fibroblasts and iPSC are collected from a healthy human, i.e., is free from PAD or other diseases. In preferred embodiments, the enhanced ECs are administered to the PAD patient by injection.

### BRIEF DESCRIPTION OF THE DRAWINGS

FIGS. 1A-1F illustrate blood flow and capillary density being improved in ischemic hindlimbs by induced endothelial cell (IEC) transplantation. FIG. 1A shows representative images of laser Doppler perfusion imaging. FIG. 1B summarizes data of perfusion ratio (value of the ischemic limb divided by that of nonischemic limb) at days 0, 7, and 14 after treatment (n=5 each group). *P<0.05, repeated-measures ANOVA followed by multiple comparisons with Bonferroni method). EGM indicates endothelial cell growth medium. FIG. 1C illustrates immunofluorescent CD31 staining of ischemic tissues from mice treated with iECs, vehicle, or human dermal microvascular endothelial cells (HMVECs). FIG. 1D illustrates quantification of total capillary density in the ischemic limbs (n=3). * P<0.05, control vs iECs; #P<0.05, control vs HMVECs, 1-way ANOVA corrected with Bonferroni method. FIG. 1E shows representative images of ischemic limbs at day 18 from iEC-, vehicle-, and HMVEC-treated animals. FIG. 1F illustrates hindlimb ischemia score obtained by 2 blinded observers (n=3). *P<0.05, control vs iECs; #P<0.05, control vs HMVECs, 1-way ANOVA corrected with Bonferroni method.

FIGS. 2A-2C illustrate IL-4/IL-13 promoting the angiogenic program of endothelial cells (ECs) by stimulating angiogenesis, proliferation and capillary assembly. FIG. 2A shows representative images and quantification of EdU incorporation of ECs from mice stimulated with vehicle, IL-4, or IL-13 (n=4; scale bars, 100 μm). FIG. 2B shows representative images and quantification of migrated ECs of mice with the stimulation of vehicle, IL-4, or IL-3 for 48 h (n=5; scale bars, 250 μm). FIG. 2C shows representative light-microscope images and tube length quantification of ECs from mice on Matrigel treated with vehicle, IL-4, or IL-13 (n=5; scale bars, 200 μm). Error bars represent mean±SEM. *p<0.05, **p<0.01, ***p<0.001, ****p<0.0001; by Two-way ANOVA test.

FIGS. 3A-3D illustrate IL-4/IL-13 promoting the angiogenic function of primary endothelial cells isolated from IL4Rαf/f while these effects are abolished by endothelial cell-specific IL4Rα knockout. Transcriptional analysis of angiogenic genes IGF-1 (FIG. 3A), VEGF-A (FIG. 3B), FGF-2 (FIG. 3C), and GM-CSF (FIG. 3D) induced by IL-4 or IL-13 treatment for 12 h (n=4). Error bars represent mean±SEM. *p<0.05, **p<0.01, ***p<0.001; ns indicates no significant difference.

FIGS. 4A-4D show administration of exogenous IL-4/IL-13 rescuing revascularization defect in diabetic ischemic hindlimb via endothelial IL-4Rα signaling. FIG. 4A illustrates the experimental scheme of development of STZ-induced diabetic model and treatment of recombinant IL-4/IL-13 following HLI injury. FIG. 4B shows Laser Doppler imaging of paw reperfusion at indicated time points (n=6). FIG. 4C illustrates the distribution of necrotic toe number at each paw 7 days post-ischemia (n=6). FIG. 4D shows representative hematoxylin and eosin staining images of gastrocnemius muscle 7 days after hindlimb ischemia (n=6; scale bars, 40 μm).

FIG. 5 illustrates the capillary density of IL-4- or IL-13-treated diabetic mice increasing nearly to the extent seen in non-diabetic mice. The capillary density was indicated by VE-Cadherin-positive staining of gastrocnemius muscle sections at day 7 after HLI induction (n=6; scale bars, 40 μm).

FIGS. 6A-6D illustrate examples of successful generation of functional endothelial cells from human induced pluripotent stem cells (iPSCs) through transdifferentiation. FIG. 6A shows iPSCs before transdifferentiation. FIG. 6B shows successful transdifferentiation of induced-endothelial cells (iECs) from iPSCs. FIG. 6C shows iECs exhibits normal ability of tube formation, demonstrating the successful transdifferentiation from iPCSs. FIG. 6D illustrates immunofluorescence staining showing that iECs expresses endothelial cell marker, CD144, illustrating the successful transdifferentiation from iPCSs. Scale bar, 50 μm.

FIGS. 7A-7D illustrate IL-4/IL-13 promoting the angiogenic program of human induced pluripotent stem cell-derived-endothelial cells (iPSC-ECs) by stimulating angiogenesis, proliferation, capillary assembly and cell viability. FIG. 7A shows representative images (left) and quantification (right) of EdU incorporation of iPSC-ECs stimulated with vehicle, IL-4, or IL-13, demonstrating that IL-4/IL-13 treatment significantly stimulates cell proliferation (n=4; scale bars, 100 μm). FIG. 7B shows representative images (left) and quantification (right) of migrated iPSC-ECs with the stimulation of vehicle, IL-4, or IL-3 for 48 h, illustrating that IL-4/IL-13 treatment remarkably stimulates the endothelial cell migration (n=7; scale bars, 500 μm). FIG. 7C shows representative light-microscope images of iPSC-ECs on Matrigel treated with vehicle, IL-4, or IL-13, demonstrating that IL-4/IL-13 treatment significantly stimulates tube formation of endothelial cells (scale bars, 1000 μm). Error bars represent mean±SEM. *p<0.05 vs. control group by Student's t-test. FIG. 7D shows cell viability of iPSC-EC treated by vehicle, IL-4 or IL-13 for 24 h (n=3), illustrating that IL-4/13 can promote cell proliferation under infertile conditions, with the cell viability ratio 119.1±9.26% (IL-4 group) and 123.7±11.16% (IL-13 group).

FIG. 8 shows blood perfusion of hindlimb ischemia in mice using laser doppler. Injection of IL-4/IL-13-treated primary mouse endothelial cells (ECs) reveals stronger angiogenic capability in driving blood reperfusion in the thigh area, in comparison with conventional cell therapy (injection of non-treated ECs). Mice are injected with ECs culture medium as a control.

FIG. 9 shows blood reperfusion in the paw area from mice using laser Doppler. Compared with those injected with non-treated ECs or the control group, the blood reperfusion is promoted by being injected with IL-4/IL-13-treated ECs.

FIG. 10 shows injection of IL-4/IL-13 treated ECs. Injection of IL-4/IL-13 treated ECs stimulates CD31 expression in the injured muscle using confocal microscopy, compared with those injected with non-treated ECs or the control group.

FIG. 11 illustrates flow cytometry analysis of muscle tissues from HLI mice injected with IL-4/IL-13-treated endothelial cells. FIG. 11 shows that injection of IL-4/IL-13 treated ECs increases the endothelial cell population in the injured muscle, compared with those injected with non-treated ECs or the control group, using flow cytometry.

15 FIG. 12 illustrates flow cytometry analysis of M2 macrophage population. FIG. 12 shows that injection of IL-4/IL-13 treated ECs increases the M2 macrophage population in the injured muscle, compared with those injected with non-treated ECs or the control group, using flow cytometry.

FIG. 13 shows muscle recovery and regeneration of myofibers. FIG. 13 shows that injection of IL-4/IL-13 treated ECs accelerates the muscle regeneration and alleviates the fibrosis within myofibers, compared with those injected with non-treated ECs or the control group, using H&E staining and Picrosirius Red staining.

FIG. 14 shows blood perfusion of hindlimb ischemia in diabetic mice using laser doppler. Injection of IL-4/IL-13-treated (ECs) reveals stronger angiogenic capability in driving blood reperfusion in the thigh area, in comparison with conventional cell therapy (injection of non-treated ECs). Mice are injected with ECs culture medium as a control.

FIG. 15 shows blood reperfusion in the paw area from diabetic mice using laser Doppler. Compared with those injected with non-treated ECs or the control group, the blood reperfusion is promoted by being injected with IL-4/IL-13-treated ECs under hyperglycemia condition.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the term “about” is used, these compositions contain the stated amount of the ingredient with a variation (error range) of 0-10% around the value (X±10%). In other contexts, the term “about” is providing a variation (error range) of 0-10% around a given value (X±10%).

As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. As used herein, the term “animal” may be, for example, humans, pigs, horses, goats, cats, dogs, apes, chimpanzees, orangutans, guinea pigs, hamsters, cows, or sheep. These benefits can include, but are not limited to, the treatment of a health condition, disease, or disorder; prevention of a health condition, disease or disorder; immune health; enhancement of the function of an organ, tissue, or system in the body. The preferred subject in the context of this invention is a human. The term does not denote a particular gender. Thus, male and female subjects are intended to be covered. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating, preventing, inhibiting, or improving a condition, disease, or disorder in a subject. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating; reducing; inhibiting; ameliorating; abatement; remission; diminishing of symptoms or delaying the onset of symptoms; slowing in the rate of degeneration or decline; making the final point of degeneration less debilitating; and/or improving a subject's physical or mental well-being or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, the terms “arresting”, “reducing”, “inhibiting”, “blocking”, “preventing”, “alleviating”, “delaying”, “forestalling”, “minimizing”, or “relieving” the onset of a particular sign or symptom of the condition, disease, or disorder. Inhibition can, but is not required, to be absolute or complete; meaning, the sign or symptom may still develop at a later time. Inhibition can include reducing the severity of the onset of such a condition, disease, or disorder, and/or inhibiting the progression of the condition, disease, or disorder to a more severe condition, disease, or disorder.

When referring to a compound or composition, or a specific cell type, “promoting”, “promote”, or promotes” means that the compound or composition increases angiogenesis and muscle regeneration by at least about 2.5%, 5%, 7.5%, 10%, 15%, 20%, 25%, 30%, 35%, 40%, 45%, 50%, 55%, 60%, 65%, 70%, 75%, 80%, 90%, or 100% compared to how the condition would normally exist without application of the compound or composition comprising the compound.

As used herein, a “pharmaceutical” refers to a compound or composition manufactured for use as a medicinal and/or therapeutic drug.

As used herein, an “isolated” or “purified” compound or composition is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, the terms “determining,” “measuring,” and “assessing,” and “assaying” are used interchangeably and include both quantitative and qualitative determinations.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

All references cited herein are hereby incorporated by reference in their entirety.

Current cell therapies rely on paracrine signalling instead of the differentiation of ECs. Endothelial cell therapies are not available for PAD in the current state. In addition, there is no existing strategy using IL-4/IL-13 to boost the endothelial cells (ECs) against the progression of PAD in either basic research or clinical trials.

The present invention relates to a novel method for treating peripheral arterial disease (PAD), where the method involves several steps, comprising: (a) collecting fibroblasts and pluripotent stem cells (iPSCs) from a healthy subject b) obtaining induced pluripotent stem cells-derived endothelial cells (iPSC-ECs) and induced endothelial cells (iECs) from the collected fibroblasts; (c) further inducing the iPSC-ECs and iECs with IL-4/IL-13 to obtain enhanced ECs; (d) administering an effective amount of the enhanced endothelial cells derived from iPSC-ECs and iECs to the PAD subject to promote angiogenesis and muscle regeneration in ischemic muscle tissue.

In some embodiments, the subject is a mammal. In preferred embodiments the mammal is a human. In embodiments, the subject is a patient. In preferred embodiments, the patient is healthy. In certain embodiments, the subject includes, but is not limited to, one or more healthy patients.

In some embodiments, the method of the subject invention replenishes the injured site with pro-angiogenic mature endothelial cells (ECs) into the injured site, thereby reconstituting the vascular network.

In other embodiments, the method of the subject invention leverages the angiogenic potential of type 2 cytokines, IL-4 and IL-13, to enhance the efficacy of induced-endothelial cells derived from human induced pluripotent stem cells and fibroblasts. This approach aims to develop novel therapies targeting PAD and its related vascular complications.

### The Cell-Based Therapy of the Present Method is Sustainable

In some embodiments, pre-treating the ECs with IL-4/IL-13, polarizes ECs into pro-angiogenic fate, improving their viability and capability during transplantation. In the past decades, studies have shown disappointing results in clinical trials that focused on over-expressing pro-angiogenic factors, indicating that intrinsic expression of these factors in injured cells fails to promote revascularization.

### The Subject Method Allows for Long-Term Release of Angiogenic Factors

In preferred embodiments, the method of the subject invention utilizing iPSC-ECs or iECs for collecting fibroblasts, is minimally invasive. The administration of functional and healthy cells to the injury site withstands the proinflammatory microenvironment and shapes the tissue niche towards regeneration. Unlike stem cells, ECs are mature cells, thereby overcoming concerns related to the use of stem cells. While the delivery of ECs has been proved in improving blood reperfusion by us and others, empowering the transplanted ECs furthers stimulating the vasculature formation with other resident ECs.

In preferred embodiments, initiation of angiogenesis in transplanted cells accelerates revascularization. In comparison, other cellular approaches need to wait for differentiation and maturation. Furthermore, the method of the subject invention is less invasive. Currently, treatment strategy against chronic limb-threatening ischemia primarily involves revascularization by surgical intervention. However, retrospective analysis suggests that neither endovascular nor open surgery improves the amputation-free survival. Alternatively, stem cells are sourced from patients or healthy donors requiring an invasive procedure.

### The Subject Method is Highly Specific, Personalized, and Lacks Pathogenicity

In more preferred embodiments, delivery of enhanced ECs directly to the injury site rebuilds the vascular network. Enriching the transplanted ECs provides a direct and reliable way to ensure vascular maturation. The transplanted cells are derived from healthy donors and/or patients. In certain embodiments, the transplanted cells are derived from the patient themselves, greatly diminishing the risk of immune responses due to graft rejection. This approach avoids the need for antigen matching or immunomodulation between donor and patients.

In some embodiments, the transplanted ECs recapitulate the functionality of bonafide ECs physiologically, including angiogenesis and arteriogenesis by tube formation. In preferred embodiments, intramuscular delivery of mature ECs does not require any viral vector, nanoparticles or lipofectamine compared with gene therapy.

In some aspects, the cellular platform provided by the subject invention can be tailored to different bioengineering approaches, such as hydrogels, patterned scaffold, immunomodulation, as well as 3D bioprinting, to maximize the therapeutic efficacy.

In some embodiments, the subject method, leveraging IL-4 and IL-13 restores blood perfusion and promotes muscle regeneration in PAD patients, ultimately improving their outcomes and quality of life. In preferred embodiments, induction with IL-4/IL-13-treated endothelial cells (enhanced ECs) promotes full muscle regeneration and about 50% revascularization in a PAD patient. In contrast, in a control group and untreated ECs injection group, revascularization is promoted by about 20%.

In some embodiments, treatment with IL-4 and IL-13 promotes a two-three fold increase in the angiogenic program of induced endothelial cells derived from human iPSCs and fibroblasts compared to the untreated group.

In other embodiments, administering of PSC-ECs and/or ECs induced with IL-4/IL-13, facilitates revascularization in patients with a BMI higher than 30 and with diabetes that suffer from PAD. This approach paves the way for the further development of personalized healthcare.

In further embodiments, treatment with IL-4/IL-13 enhances endothelial cells contributing to relieving the vascular complications of PAD patients and decreasing their amputation risks. In certain embodiments, induction with IL-4/IL-13-treated endothelial cells (enhanced ECs) fully restores blood perfusion, promotes revascularization by about 50%, increases capillary density by about 50%, and fully restores the suppressed angiogenic program. For reference, the angiogenic program is 50% suppressed before treatment.

In more preferred embodiments, delivering IL-4/IL-13 improved ECs to the injured sites restores blood perfusion in ischemia muscles and avoids potential health concerns including tumor induction. The direct transplantation of ECs derived from iPSCs or fibroblasts has been proven to restore blood perfusion in animals with PAD by our previous studies. Considering the high prevalence of PAD in diabetic patients, transplanted ECs may be also susceptible to the chronic inflammation induced by ischemia and diabetes, holding promise for promoting and optimizing personalized healthcare. Moreover, the use of IL-4/IL-13 offers protection to the ECs prior to transplantation, which enhances the efficacy of the cell therapies. The present invention offers cell variability and vasculogenic potential in transplanted cells (i.e., ECs) at the injured site.

### Materials and Methods

Human iPSC-EC Generation

Human iPSCs were cultured until they reach 90% confluence. Subsequently, these cells were incubated in differentiation medium, which consisted of Advanced DMEM/F12 supplemented with Wnt agonist CHIR 99021 (5 μmol/L), bone morphogenetic protein-4 (BMP4, 25 ng/ml), B27 supplement, and N2 supplement. This differentiation process lasted 3 days. The cells were then dissociated using HyQtase and seeded in StemPro media, supplemented with forskolin (5 μmol/L), vascular endothelial growth factor (VEGF, 50 ng/mL), and polyvinyl alcohol (2 mg/mL) for 4 days. Subsequently, the cells were rinsed with PBS and cultured in endothelial growth media supplemented with an additional VEGF (100 ng/ml) for an additional 5 days. In addition, we utilized a second method to generate human iPSC-EC. After reaching 90% confluence, cells were incubated in differentiation medium, which consisted of Knockout DMEM supplemented with Wnt agonist CHIR 99021 (6 μmol/L), bone morphogenetic protein-4 (BMP4, 40 ng/ml), and vascular endothelial growth factor (VEGF, 20 ng/mL). This differentiation process lasted 2 days. The cells were then induced by EGM-2 medium, supplemented with transforming growth factor-8 (TGF-8) kinase inhibitor SB-431542 (10 μmol/L), fibroblast growth factor-2 (FGF-2, 25 ng/mL), and VEGF (50 ng/mL) for 4 days. Throughout this process, a constant temperature of 37° C. and 5% CO2 in a humidified incubator was maintained. To obtain purified ECs, fluorescence-activated cell sorting was utilized on the pluripotent stem cell-derived populations.

Cell Treatment of iPSC-ECs

After obtaining the iPSC-ECs, we proceeded with stimulating their angiogenic potential using IL-4/IL-13. 10 ng/mL was applied to assess their angiogenic capabilities. The replicative capability was measured by EdU staining assay according to the manufacturer's instructions (Cat #C0078L, Beyotime Biotechnology, China). Briefly, the iPSC-ECs were treated with either IL-4 or IL-13 for 24 hours, followed by EdU labelling at 37° C. for 6 hours. To fix and denature the cells, a fixing-denaturing solution was introduced, followed by a 15-minute incubation. A staining working solution was then added to the cells and incubated at room temperature for 30 minutes. After washing, the nuclei were stained with DAPI solution for 5 min. Finally, the immunofluorescent signals were detected and measured by Leica TCS SP8 Confocal Microscope System, and the EdU-positive cells were counted by Image J software.

### Human iEC Generation

To generate induced endothelial cells (iECs) from fibroblasts, we followed our previously established protocol. Human primary HJ fibroblasts derived from foreskin dermal tissue were cultured in DMEM supplemented with 10% fetal bovine serum (FBS), 1 mmol/L L-glutamine, and 1% non-essential amino acids. Lentiviral vectors encoding ETV2, FLI1, GATA2, and KLF4 were transduced into the fibroblasts. Following transduction, the fibroblasts were maintained overnight on gelatin-coated dishes in DMEM supplemented with 10% FBS. For induction of differentiation, the fibroblasts were treated with differentiation medium composed of DMEM supplemented with 20 ng/mL BMP4, 50 ng/mL VEGF, and 20 ng/mL bFGF for a duration of 3 days. Subsequently, the differentiation medium was replaced with EC growth medium EGM-2 supplemented with 10 μmol/L SB341542 (a TGF-β receptor kinase inhibitor) until day 14, facilitating fluorescence-activated cell sorting for cell purification. The sorted cells were expanded in EGM-2 supplemented with 10 μmol/L SB341542 for a total duration of 28 days, allowing for a further expansion and maturation of the iEC population.

### Murine ESC-ECs Induction

The murine embryonic stem cells were placed in ultralow non-adhesive dishes and cultured in a differentiation medium composed of α-Minimum Eagle's Medium, 10% FBS, 1% penicillin/streptomycin, and 0.05 mmol/L β-mercaptoethanol. After 4 days of suspension culture, the embryoid body aggregates were transferred to 0.2% gelatin-coated dishes and cultured in the same differentiation medium. Following a period of 3 weeks, the cells have undergone purification using fluorescence-activated cell sorting FACS with an anti-mouse VE-cadherin or CD31 antibody.

### Cell Treatment of ESC-ECs

After obtaining the mature ECs, we proceeded with stimulating their angiogenic potential using a range of IL-4/IL-13 doses. Various concentrations, including but not limited to 1 ng/ml, 5 ng/mL, 10 ng/mL, 25 ng/mL, 50 ng/ml, and 100 ng/mL, of IL-4 or IL-13 were applied to assess their angiogenic capabilities. These treated-endothelial cells were referred as ‘enhanced ECs’.

### CCK8 Cell Viability Assay

iPSC-ECs were seeded on 96-well plate at a density of 5×103 cells per well. Following the cells attached to the plate, specific conditional treatment medium (in 1% FBS-supplemented EGM-2 medium) was applied to replace the culture medium and the cells were incubated for 24 h. After adding 10 μl CCK8 solution (Cat #C0038, Beyotime Biotechnology, China) to each well, cells were incubated for 2 h and the absorbance was measured at 450 nm wavelength.

### Peripheral Arterial Disease (PAD) Model

To assess the viability, localization, and therapeutic potential of iPSC-ECs or iECs, we utilized a murine model of hindlimb ischemia, according to the methodologies outlined in our previous publication. The 10⁶ human enhanced ECs were administered intramuscularly (IM) into the ischemic hindlimbs of NOD/SCID/IL2Rγ-mice following excision of the femoral artery. The ligation of femoral artery was undergone in these obese mice and their counterparts. The 10⁶ murine enhanced ECs were later injected into the ischemic muscle. Similarly, a diabetic model was developed by using streptozotocin while the PBS injection is used as a vehicle control. The femoral artery from these diabetic and normoglycemic mice was excised to develop the PAD model. The 10⁶ murine enhanced ECs were then administered into the injured muscle.

### Outcome Measurements for Studying the Pro-Angiogenic Effect of Enhanced ECs: Endothelial Marker Expression

The expression of the key EC markers was evaluated, including but not limited to KDR, CD31, VE-cadherin, vWF, and eNOS, using a combination of quantitative PCR, western blot, and immunofluorescence techniques.

### Replicative Capability

The iPSC-ECs or iECs were treated with either IL-4 or IL-13 for 48 hours. Following this treatment, BrdU labeling solution was added to the culture medium, and the cells were incubated at 37° C. for 3 hours. To fix and denature the cells, a fixing-denaturing solution was introduced, followed by a 30-minute incubation. An anti-BrdU-POD (conjugated with peroxidase, POD) working solution was then added to the cells and incubated at room temperature for 90 minutes. After washing, a substrate solution was added, and the cells were incubated for 30 minutes. Finally, the absorbance at 450 nm was measured using a plate reader. Besides, we employed the xCELLigence instrument (Roche) to assess proliferation. The instrument was placed in a humidified incubator set at 37° C. and 5% CO2. iPSC-ECs or iECs were harvested, and a standardized number of cells (ranging from 3000 to 5000) was seeded into 16-well plates (E-plate, Roche) with or without IL-4/IL-13 treatment. The impedance value of each well, representing the Cell Index (CI) value, was continuously monitored by the xCELLigence system. CI values were recorded every minute during the initial 4 hours and then every hour for the remaining days.

### Telomere Length

Monochrome Multiplex PCR (MMqPCR) technique was employed. Cellular DNA was isolated using the Qiagen® AllPrep® DNA/RNA/Protein Mini Kit. PCR tubes were used to contain the samples and standards. The standard was prepared through a two-fold serial dilution. A PCR mix was added to each tube and thoroughly mixed. The resulting samples and standards were transferred to a white LightCycler® 480 Multiwell plate (384 wells), with 10 μL of the total PCR preparation mix being pipetted into each well. PCR runs were conducted using a Roche LightCycler® 480 instrument (Software: 1.5.1.62) with a preinstalled MMqPCR protocol. The raw data obtained from the PCR runs were subjected to preprocessing using a Python script. This script enabled the differentiation between the telg/telc primers and the single-copy gene (SCG) primer (human β-globin). After preprocessing, the data was re-uploaded to the Roche LightCycler® system to calculate the T/S ratio, which represents telomere length.

### Cell Migration

iPSC-ECs or iECs were pre-stimulated with IL-4 or IL-13, these cells (2×10⁴ cells) were subsequently seeded onto the upper chamber of individual transwell inserts. These inserts were then placed within EGM-2 medium supplemented with 20% FBS. Following a migration period of 48 hours, the cells that have migrated to the lower side of the membrane were identified and labeled through crystal violet staining. In addition, we utilized a wound healing scratch assay as a second method to evaluate cell migration. iPSC-ECs or iECs were cultured in a 12-well dish until reaching confluency. Moreover, we utilized a third method to evaluate cell migration. iPSC-ECs were seeded onto the upper chamber of individual transwell inserts with blank Endothelial Cell Growth Medium-2 (EGM-2, Lonza, Cat #CC-3162). Subsequently, a gap of approximately 0.5 mm was created by carefully scraping along the surface of the dish. IL-4/IL-13 was applied to stimulate the cells for 2 hours and then the treatment medium was washed out. The migration of cells across the gap was monitored and recorded over a specific time period.

Uptake of ac-LDL

iPSC-ECs or iECs have undergone a 4-hour incubation with Dil-labeled ac-LDL (10 μg/ml) at 37° C. After the incubation period, the cells were washed using 1× PBS to remove any unbound ac-LDL. Subsequently, the cells were visualized and photographed under a fluorescence microscope to observe the uptake of Dil-labeled ac-LDL.

### Nitric Oxide (NO) Release

To evaluate the ability of iPSC-ECs or iECs to produce NO, we quantified intracellular nitric oxide in the ECs using NO specific probe 4,5-diaminofluorescein-2-diacetate (DAF-2DA) and flow cytometry.

### Tube Formation

A total of 2.5×10⁵ iPSC-ECs or iECs cells were seeded onto 24-well or 96-well plates that have been pre-coated with growth factor-reduced Matrigel. Matrigel provided a supportive matrix mimicking the extracellular environment necessary for tube formation. Subsequently, the plates were incubated at 37° C. for a period of 24 hours to allow the cells organizing and forming capillary-likestructures. After the incubation period, images capturing the tube formation was acquired using a light microscope.

### Primary Mouse Endothelial Cells Isolation and Culture

Primary endothelial cells were isolated from mice's lungs using 5 U/ml Neutral Protease (Worthington, Cat #LS02109) followed by affinity selection with CD31 MicroBeads (Miltenyi Biotec, Cat #130-097-418). Isolated primary endothelial cells were cultured in Endothelial Cell Growth Medium-2 (Lonza, Cat #CC-3162) and maintained at 37° C. with 21% O₂ and 5% CO₂. Cells were seeded in 6-well plate before treatment. Cells were treated with IL-4 (10 ng/ml) or IL-13 (10 ng/mL) for 24 hours. After treatment, cells were harvested with 0.5% trypsin and resuspended in Endothelial Cell Growth Medium-2.

### Hindlimb Ischemia (HLI) Model and Cell Injection

Mice were anesthetized with 75 mg/kg ketamine and 10 mg/kg xylazine. Following the introduction of a small incision on the skin of the hindlimb, iliac artery was ligated with 6-0 silk sutures. Thereafter, the overlying skin was closed with 4-0 silk sutures. Male mice were used across this study.

Untreated primary endothelial cells, IL-4-treated endothelial cells and IL-13-treated endothelial cells were harvested using 0.5% Trypsin and subsequently resuspended with Endothelial Cell Growth Medium-2. Then, cells (1×10⁶) were intramuscularly injected into mice after iliac artery ligation. Endothelial Cell Growth Medium-2 was intramuscularly injected into mice as a vehicle control.

### Angiogenic Cytokines Expression

Human Angiogenesis Proteome Profiler™ antibody arrays (R&D Systems) were employed. Human iPSC-ECs or iECs were cultured either under hypoxia (1% O2) or normoxia (21% O₂) for 24 hours. The conditioned media from each well were collected and filtered using 0.2 μm sterile filters to remove any debris or cells. The filtered conditioned medium was then incubated with an antibody cocktail (1:1000) at room temperature for 1 hour. The antibody cocktail contained antibodies specific to various angiogenic cytokines. Nitrocellulose membranes containing capture antibodies were used for the antibody array. These membranes were blocked using an assay-specific blocking solution to prevent non-specific binding. Subsequently, the sample/detection antibody cocktail mixture was added to the membranes and incubated overnight at 4° C. on a rocking platform. Streptavidin-HRP (1:2000) was later added to the membranes and incubated for 30 minutes at room temperature. Streptavidin-HRP bound to biotinylated antibodies present in the detection antibody cocktail, enabling the visualization of captured cytokines. The membranes were washed to remove any unbound reagents and then incubated with ECLplus (Amersham, Buckinghamshire, UK). After the appropriate incubation time, the membranes were exposed to X-ray film for 3 minutes to develop the chemiluminescent signal. Quantification of the array data was performed using Image J software through densitometry analysis. This analysis allowed for the measurement of the signal intensity of each captured cytokine spot on the membrane.

### Matrigel Plug In Vivo

In this assay, we assessed the therapeutic potential of iPSC-EC or iEC cells by implanting them subcutaneously into the abdominal region along with Matrigel. The implanted cells within the Matrigel created a favorable microenvironment for neovessel formation over a period of 2 weeks. Some of these vessels might establish connections with host vessels. For each implantation, a total of 0.5×106 cells were embedded in 0.5 ml Matrigel supplemented with 200 ng/mL basic fibroblast growth factor. Subsequently, the cell-Matrigel mixture was injected subcutaneously into SCID mice, with two plugs being implanted in each animal. SCID mice were used to minimize immune rejection and provide a permissive environment for the survival and growth of the implanted cells. Over a duration of 2 weeks, the plugs allowed the iPSC-ECs or iECs to interact with the surrounding environment, promoting neovessel formation and potential integration with host vessels. After 2 weeks, the plugs were explanted to enable histological analysis of vessel formation. This analysis helped us to evaluate the extent and quality of neovessel formation within the Matrigel. To differentiate human vessels from native murine vessels, we utilized human-specific antibodies targeting endothelial cells, such as CD31, to identify human-specific vessels. This identification was crucial for determining the contribution of the implanted iPSC-ECs or iECs to vessel formation. Similarly, mouse-specific endothelial cell antibodies were employed to identify murine-specific vessels, aiding in the characterization of the host vasculature. For this experiment, 14 to 16-week-old SCID mice were subjected to the implantation of Matrigel plugs. These plugs contained either non-treated iPSC-ECs/iECs or IL-4/IL-13-pre-treated iPSC-ECs/iECs. To ensure the specific targeting of cytokines to ECs, a thorough washout of IL-4/IL-13 was performed prior to implantation.

Outcome Measurements for Studying the Therapeutic Effect of Enhanced ECs in Animals: Transduction of iPSC-ECs And iECs with Double Fusion Receptor Construct

To enable noninvasive tracking of the purified iPSC-ECs or iEC cells in vivo, cells were transduced with a lentiviral vector carrying a double fusion reporter construct driven by an ubiquitin promoter. This construct comprised firefly luciferase (fluc) and enhanced green fluorescence protein (GFP) under the control of the promoter. After transduction, the cells expressing GFP were isolated using fluorescence-activated cell sorting and maintained in differentiation medium supplemented with 50 ng/mL vascular endothelial growth factor. To establish a correlation between cell density and fluc activity, the transduced cells were incubated with a reporter probe called d-luciferin (150 g/mL). Live animal bioluminescence imaging was performed using the PerkinElmer IVIS Lumia III in vivo imaging system to examine the BLI signals emitted by the cells. The intensity of BLI signals was quantified in units of photons/cm²/second/steradian (p·cm−2·s−1·sr−1). In addition to monitoring fluc activity, the transduced cells were undergone analysis by FACS and immunofluorescence to confirm GFP expression and verify the maintenance of an EC phenotype. This analysis involved assessing dual expression of GFP and VE-cadherin, an endothelial cell marker.

### Optical BLI of Cell Survival and Localization

To evaluate the survival and localization of the transplanted cells, optical BLI was conducted at designated time points over a period of 4 weeks. This non-invasive imaging technique allowed us to track the bioluminescent signal emitted by the cells in vivo. For this purpose, a reporter probe called d-luciferin (375 mg/kg body weight) was injected into the peritoneum of the animals. The administered d-luciferin was metabolized by the cells expressing firefly luciferase (fluc), resulting in the emission of light. The BLI analysis was performed using the Living Image software (Caliper Life Sciences). By performing optical BLI at specific time intervals, we monitored the fate of the cells over the course of the study, gaining insights into their viability, migration, and persistence within the hindlimb ischemia model.

### Laser Doppler Imaging of Blood Reperfusion

The RFLSI III Laser Speckle Imaging System (RWD Life Science) was employed to assess blood flow in both the ischemic and nonischemic hindlimbs at specified time points, following the methodology we outlined in a previous publication. This non-invasive imaging technique allowed us to evaluate the perfusion status of the hindlimbs. To ensure consistent measurements, each animal was prewarmed to achieve a core temperature of 37° C. Hindlimb blood flow was measured before and after the surgical procedure on day 0, as well as on days 3, 7, 14, 21, and 28. This longitudinal assessment enabled us to monitor the changes in blood reperfusion over the course of the study. The level of reperfusion in the hindlimb blood flow was quantified by expressing it as the ratio of the ischemic limb to the nonischemic limb. By comparing the blood flow between the two limbs, we assessed the effectiveness of the therapies in promoting revascularization and restoring blood supply to the ischemic region.

In addition, we utilized a second method to assess blood flow. Blood flow perfusion was monitored at pre-HLI injury, post-HLI injury, at day 3, day 7, and day 14 after HLI injury using RFLSI III Laser Speckle Imaging System (RWD Life Science). Blood perfusion of paw area was monitored at pre-HLI injury, post-HLI injury, and at day 14 after HLI injury using RFLSI III Laser Speckle Imaging System.

The utilization of the RFLSI III Laser Speckle Imaging System provided us with reliable and objective measurements of hindlimb blood flow, allowing for accurate and quantitative evaluation of the therapeutic interventions. These assessments contribute to our understanding of the functional outcomes and efficacy of the iPSC-ECs or iECs in promoting reperfusion and tissue recovery in the hindlimb ischemia model.

### Diabetes Mellitus Induction

Mice were introduced to diabetes mellitus by intraperitoneal injections of streptozotocin (Beyotime, Cat #Y271563) at 40 mg/kg for 5 consecutive days. Before intraperitoneal injections, mice were fasted for 4 hours and normal water was provided. After intraperitoneal injections of streptozotocin, mice were given 10% sucrose water. On experimental day 21, blood glucose level at ad libitum conditions was accessed. The development of diabetic mellitus was validated for further study when their blood glucose levels were >200 mg/dl.

### Immunofluorescence Staining

Gastrocnemius muscle samples were harvested and immediately fixed in 4% PFA after dissection. Muscle samples were embedded in paraffin and cut at the belly of the muscle into 5 um sections. Gastrocnemius muscle sections were fixed with chilled methanol at room temperature for 5 min, washed 3 times with PBS, and subsequently blocked with 10% Normal Goat Serum (Thermo Scientific, Cat #50062Z) for 30 min. Thereafter, muscle sections were incubated with primary antibody (CD31, Novus Biologicals, Cat #NB100-2284) in blocking buffer at 4° C. overnight. Samples were then washed with PBS for 3 times and incubated with secondary antibodies (Doney anti-Rat Alexa Fluor 594, Thermo Fisher, Cat #A-11007) in blocking buffer for 90 min. Nuclei were stained with Hoechst 33342 solution (Thermo Scientific, Cat #62249) and slides were embedded in Fluoromount Aqueous Mounting Medium (Sigma-Aldrich, Cat #F4680). Images were captured by Lecia SP8 confocal microscopy.

### H&E Staining

Gastrocnemius muscle samples were harvested and immediately fixed in 4% PFA after dissection. Muscle samples were embedded in paraffin and cut at the belly of the muscle into 5 um sections. Muscle sections were collected and stained with hematoxylin and eosin. Images were taken on Carl Zeiss PALM Inverted Microscope. Regeneration area was identified by the presence of central nucleated muscle fiber.

### Flow Cytometric Analysis

Muscle samples were cut into small pieces and subsequently digested with 800 U/mL Type 2 Collagenase (Worthington, Cat #LS004177) and 1 U/mL Neutral Protease (Worthington, Cat#LS02111) for 60 min. After digestion, 40 μm cell strainer (Corning, Cat #352340) were used to filter the digested muscle pieces to obtain single cell suspension. Cells were incubated with LIVE/DEAD Fixable Aqua (Invitrogen, Cat #L34965) and Fc-gamma receptors blocking antibody Anti-CD16/32 (Biolegend, Cat #101329) in FACS buffer for 25 min prior to the surface staining. Thereafter, cell surface antigens were stained with corresponding primary antibodies (CD45, CD31, F4/80). Samples were processed by Flow Cytometer (BD FACSVerse) and data were analyzed using FlowJo 10 software.

### Picrosirius Red Staining

Gastrocnemius muscle samples were harvested and immediately fixed in 4% PFA after dissection. Muscle samples were embedded in paraffin and cut at the belly of the muscle into 5 um sections. Slides underwent dewax process through twice 100% Xyline (5 minutes), 100% ethanol (1 minute), 95% ethanol (1 minute), 75% ethanol (1 minute), 50% ethanol (1 minute), 30% ethanol (1 minute), and 1 minute water. Then slides were stained with Sirius Red staining solution for 1 hour. Subsequently, the slides were destained with 0.1% acetic acid for 3 seconds, followed by 95% ethanol (3 seconds), twice 100% ethanol (2 minutes), ethanol/xyline (1:1, 2 minutes), and twice 100% Xyline (2 minutes). Slides were embedded in Fluoromount Aqueous Mounting Medium (Sigma-Aldrich, Cat #F4680). Images were captured using Carl Zeiss PALM Inverted Microscope.

Incorporation into Capillary

To evaluate the integration of exogenous cells into microvasculature of the ischemic tissue, double immunofluorescence staining for CD31 and GFP was performed. Muscle sections from each animal were subjected to this staining protocol. Capillary density was quantified using ImageJ software, which involved counting CD31+ capillary. To assess the contribution of the transplanted cells to capillary formation, the number of capillaries expressing both GFP and CD31 was counted. This dual expression indicated the successful incorporation of the transplanted cells into the existing capillary network. The densities of total capillaries and GFP+ capillaries were calculated as the number of capillaries per square millimeter (mm2), providing quantitative information on the extent of capillary formation facilitated by the transplanted cells. In addition to immunofluorescence staining, flow cytometry analysis was conducted to examine the CD45− CD31+ endothelial cell population.

### Angiogenic Program Activity

Muscle tissue samples were collected to investigate the expression of angiogenic programs within the ischemic muscle. To analyze the mRNA levels of key angiogenic factors, including, but not limited to vascular endothelial growth factor (VEGF), fibroblast growth factor (FGF), platelet-derived growth factor (PDGF), hepatocyte growth factor (HGF), and angiopoietin, quantitative PCR was employed.

### Muscle Regeneration

Gastrocnemius muscle samples were promptly fixed in 4% PFA following dissection. Subsequently, the fixed muscle samples were embedded in paraffin and sliced into 10 μm sections at the belly of the muscle. Hematoxylin and eosin staining wase performed on these muscle sections. To capture images of the stained sections, a Carl Zeiss PALM Inverted Microscope was utilized. The regenerating area was identified by the presence of centrally nucleated muscle fibers, while the area of inflammatory cell infiltration was determined by the presence of mononuclear cell infiltrates within the muscle interstitial space.

### Mechanism of Action Studies

To elucidate the mechanism by which IL-4/IL-13-treated iPSC-ECs and iECs enhance vascular regeneration, we explored how IL-4/IL-13-treated iPSC-ECs and iECs contribute to vascular regeneration through paracrine signaling and integration into the host vasculature. To analyze the cytokines secreted by the enhanced ECs, we conducted the proteomic analysis. By comparing their secretion profiles to the angiogenic profile of primary arterial endothelial cells, as we have previously done, we gained insights into the specific paracrine factors involved in the enhanced vascular regeneration mediated by IL-4/IL-13-treated iPSCs and iECs. These investigations provided a comprehensive understanding of the underlying mechanisms through which IL-4/IL-13-treated iPSC-ECs or iECs exerted their regenerative effects, facilitating the development of novel therapeutic strategies for promoting vascular regeneration.

Safety of IL-4/IL-13 Treated iPSC-ECs and iECs

Comprehensive genetic and epigenetic studies were conducted to evaluate the safety profile of these cells. This analysis included investigations to exclude any aberrations in copy number variations, mitochondrial DNA sequence, and gene promoter methylation. Furthermore, the stability of the cell phenotype following in vivo delivery was examined to ensure the maintenance of desired characteristics. Tumorigenicity studies were performed to assess and confirm the absence of tumor formation associated with the administered cells. We also analyzed additional assays to assess the overall health of the animals at the organismal level. These assays provided a comprehensive evaluation of the safety and well-being of the animals following the administration of IL-4/IL-13 treated iPSC-ECs or iECs. The specific assays to be performed included body weight monitoring, hematological analysis, organ histopathology, and physical examination (e.g., visible signs of distress, abnormalities, or adverse reactions in the animals).

### Outcome Measurements for Studying the Metabolic Benefits of Transplanting Enhanced ECs: Body Weight

The weight of the mice was regularly recorded on a weekly basis throughout the experiment.

### Glucose Level

To confirm the development of diabetes before inducing hindlimb ischemia, random blood glucose levels was examined. Additionally, an oral glucose tolerance test (OGTT) was conducted. Prior to the OGTT, mice underwent a 12-hour fasting period. After measuring the fasting blood glucose level, a dose of 2 mg glucose per gram of mouse weight (administered as a 10% glucose solution) was orally administered via gavage. Subsequently, blood glucose levels were measured and recorded at 15, 30, 60, and 120 minutes following the glucose administration.

### Inflammatory Gene Activity

Inflammatory gene expression was assessed using quantitative PCR to detect the mRNA levels of various genes, such as TNF-α, IL-1B, MCP-1, CXCL2, IL-8, IL-6, VCAM-1, ICAM-1, E-selectin, and P-selectin. Muscle tissue samples collected from the abovementioned mice were used for this investigation.

### Circulating Biomarkers

Blood plasma was used for the analysis of circulating biomarkers. Several parameters were measured, including lipid profile (total cholesterol, non-HDL cholesterol, and triglyceride) by colorimetric assays. Cytokines (IL-1B, IL-6, IL-8, MCP-1) were measured using bead-based immunoassays and detected with a flow cytometer. Additionally, circulating soluble adhesion molecules (sVCAM-1, SICAM-1, sE-selectin) were detected using ELISA.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1

Methodology for inducing endothelial cells from human induced pluripotent stem cells (iPSCs) (iPSCs): initially, human iPSCs are cultured until they reach 80% confluence. Subsequently, the cells were incubated in differentiation medium which consists of Advanced DMEM/F12 supplemented with the following components: Wnt agonist CHIR 99021 (5 μmol/L), bone morphogenetic protein-4 (BMP4, 25 ng/ml), B27 supplement, and N2 supplement. This differentiation process was conducted for three days. The cells were then dissociated using HyQtase and seeded in StemPro media, supplemented with forskolin (5 μmol/L), vascular endothelial growth factor (VEGF, 50 ng/mL), and polyvinyl alcohol (2 mg/mL) for four days.

Subsequently, the cells were rinsed with phosphate-buffered saline (PBS) and cultured in endothelial growth media supplemented with an additional VEGF (100 ng/ml) for an additional five days. Throughout this process, a constant temperature of 37° C. and 5% CO₂ in a humidified incubator was maintained. To obtain purified ECs, fluorescence-activated cell sorting was utilized on the pluripotent stem cell-derived populations.

Methodology for deriving endothelial cells from fibroblasts. Human primary BJ fibroblasts derived from foreskin dermal tissue were cultured in DMEM supplemented with 10% fetal bovine serum (FBS), 1 mmol/L L-glutamine, and 1% non-essential amino acids. Lentiviral vectors encoding ETV2, FLI1, GATA2, and KLF4 were transduced into the fibroblasts. Following transduction, the fibroblasts were maintained overnight on gelatin-coated dishes in DMEM supplemented with 10% FBS. For induction of differentiation, the fibroblasts were treated with differentiation medium composed of DMEM supplemented with 20 ng/mL BMP4, 50 ng/ml VEGF, and 20 ng/mL bFGF for a duration of three days. Subsequently, the differentiation medium was replaced with EC growth medium EGM-2 supplemented with 10 μmol/L SB341542 (a TGF-β receptor kinase inhibitor) until day 14, facilitating fluorescence-activated cell sorting for cell purification. The sorted cells were then expanded in EGM-2 supplemented with 10 μmol/L SB341542 for a total duration of 28 days, allowing for a further expansion and maturation of the iEC population.

This contribution guarantees the quality and quantity of induced endothelial cells to fulfil the requirement of cell-based therapy for the treatment of peripheral arterial diseases.

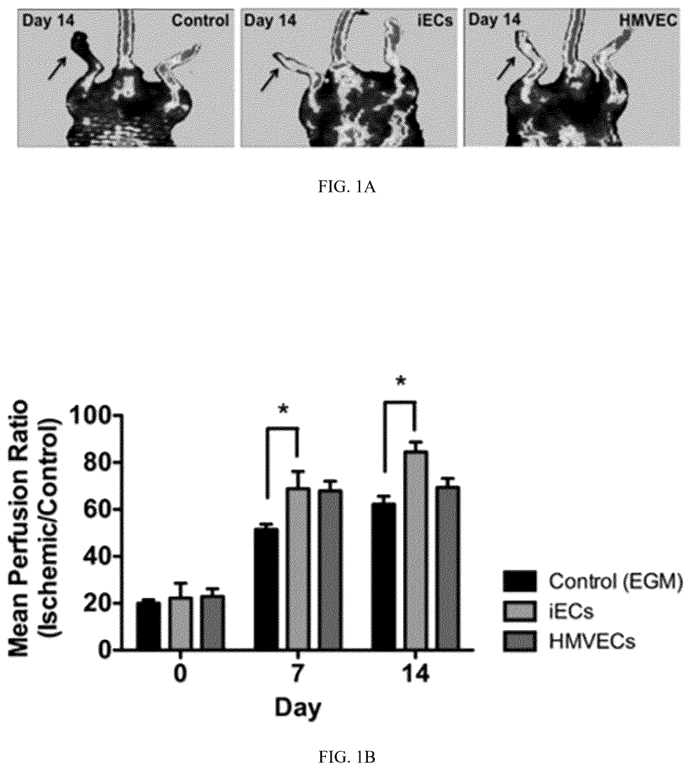

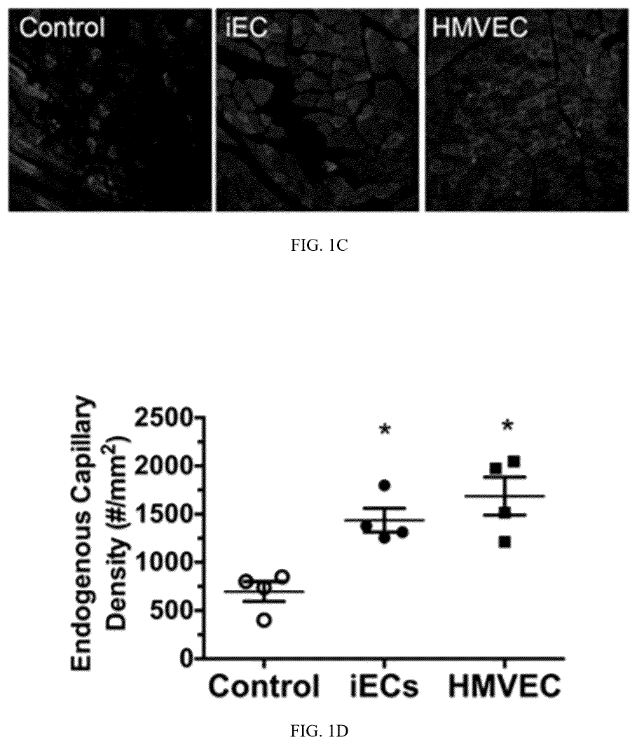

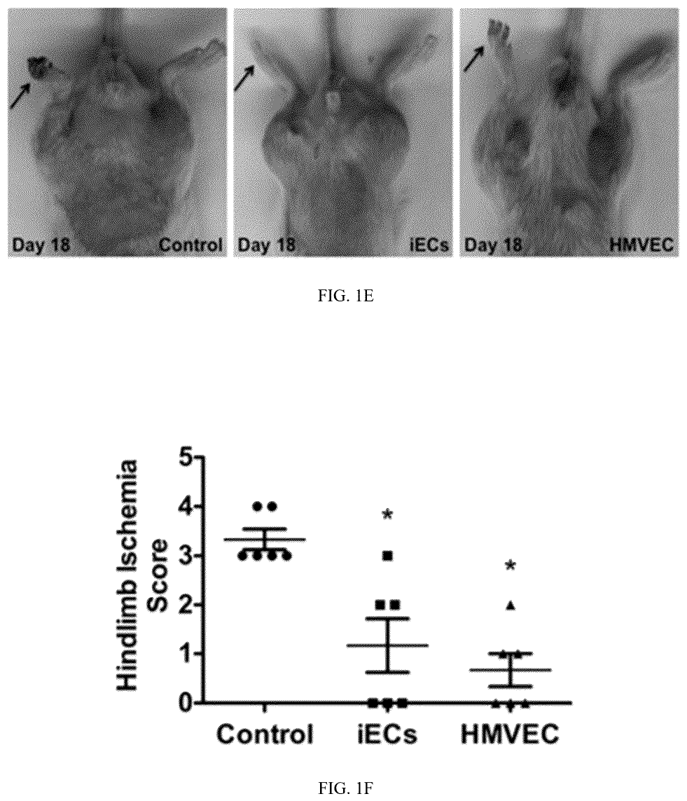

Our previous research demonstrated the therapeutic potential of stem cell therapies in enhancing blood perfusion in a murine model of peripheral arterial diseases, utilizing induced endothelial cells derived from human fibroblasts (FIGS. 1A-1F).

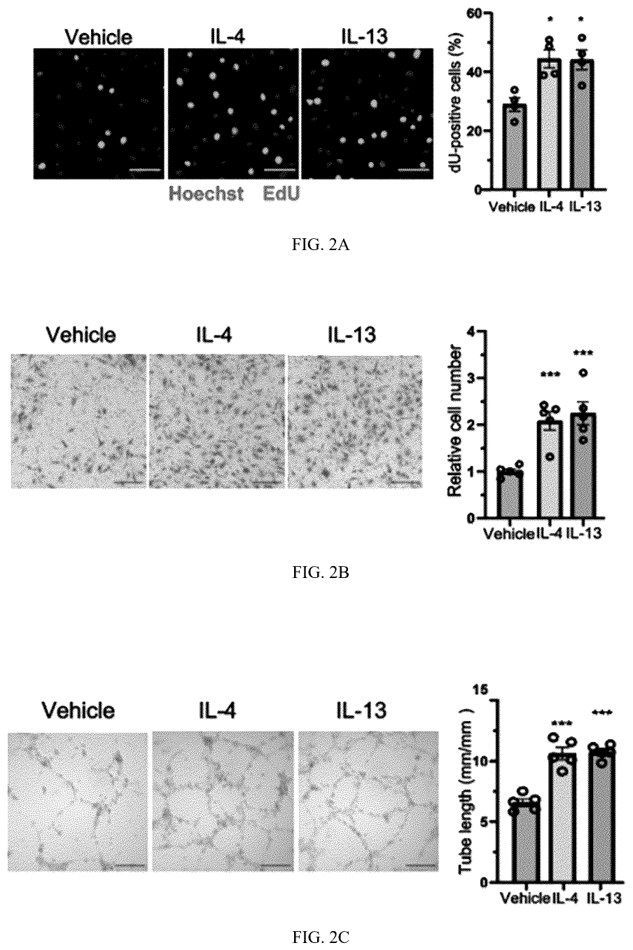

Our new research emphasized the indispensable role of type 2 cytokines, IL-4 and IL-13, in the process of revascularization in peripheral arterial diseases. Our findings highlighted the direct interaction of IL-4 and IL-13 on endothelial cells by stimulating angiogenesis, proliferation and capillary assembly, emphasizing the high feasibility of utilizing IL-4 and IL-13 to enhance the cell-based therapies for the treatment of peripheral arterial diseases (FIGS. 2A-2C).

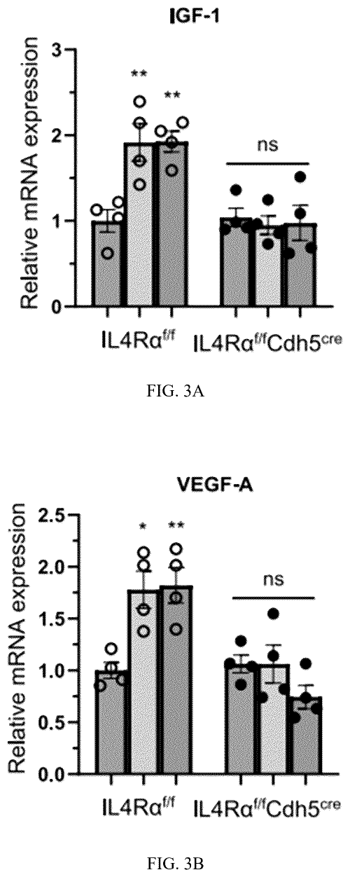

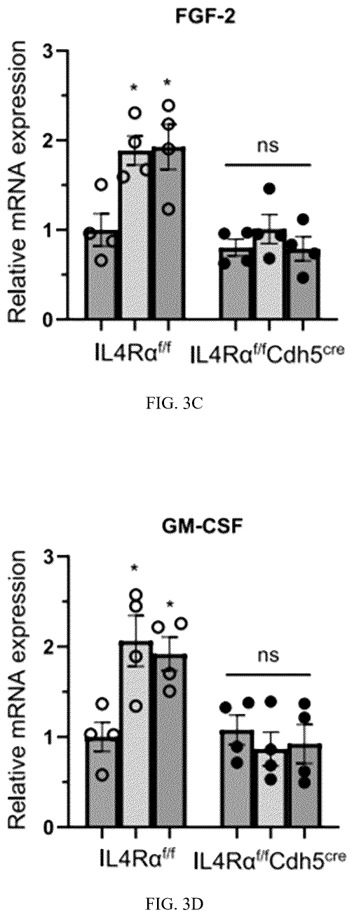

We further illustrated that the treatment of IL-4 and IL-13 promoted angiogenic function in endothelial cells. IL-4 and IL-13 increased mRNA expression levels of genes IGF-1, VEGF-A, FGF-2, and GM-CSF in endothelial cells after 12 hours treatment (FIGS. 3A-3D).

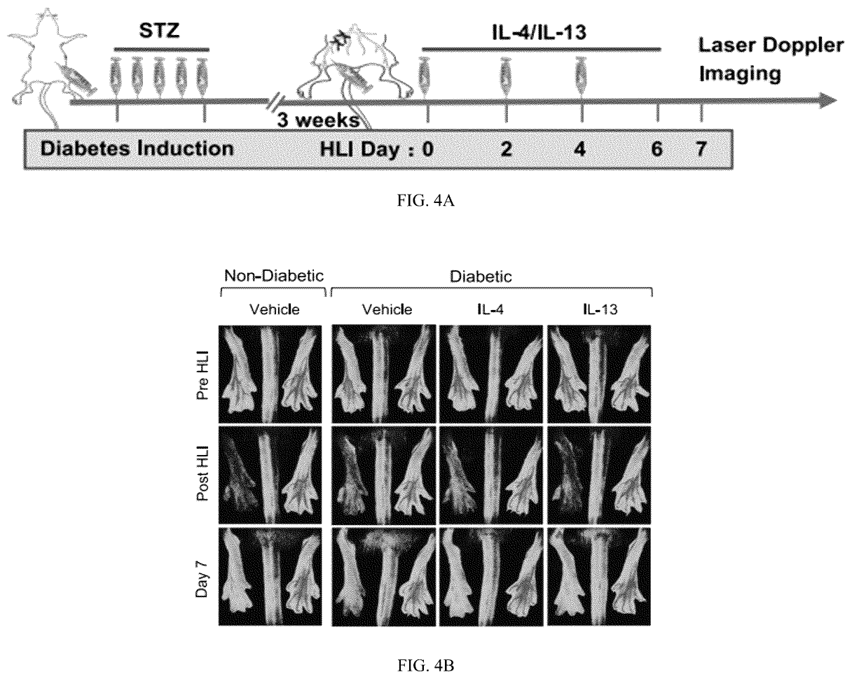

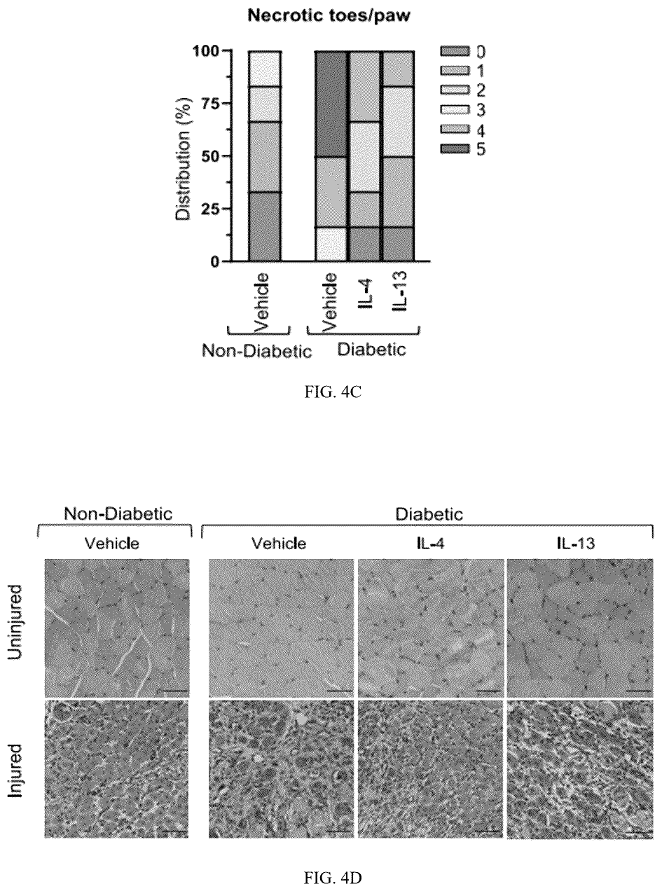

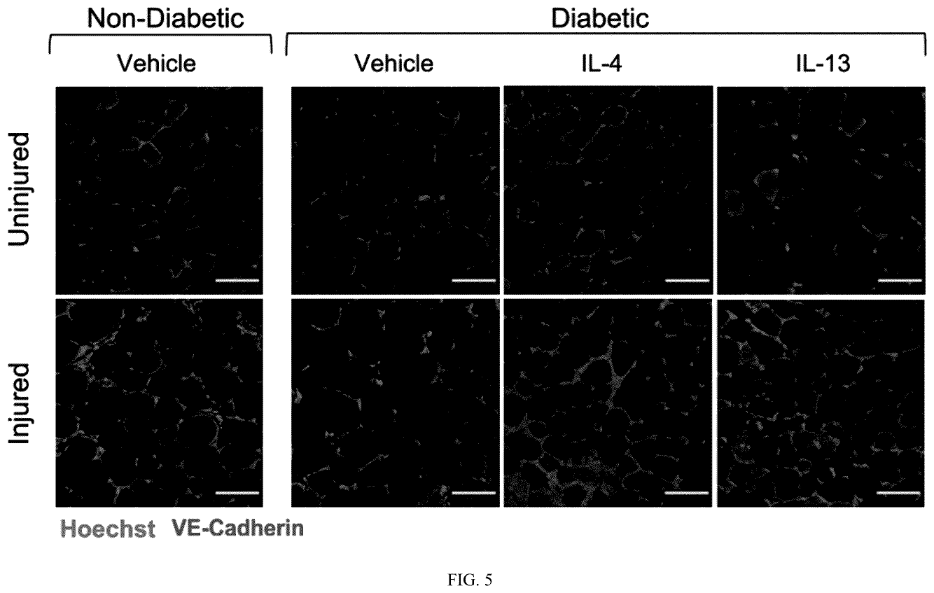

Then, we showed that exogenous IL-4/IL-13 rescued revascularization defect in diabetic ischemic hindlimb. The blood flow of paws of IL-4-and IL-13-treated mice was restored to the same extent as the non-diabetic mice. The necrotic toe number was half decreased by treatment of IL-4 and IL-13 compared to the diabetic mice. Muscle regeneration was initiated by IL-4 and IL-13 treatment. (FIGS. 4A-4D).

Additionally, we showed that the capillary density of IL-4- or IL-13-treated diabetic mice increased to the extent seen in non-diabetic mice. (FIG. 5).

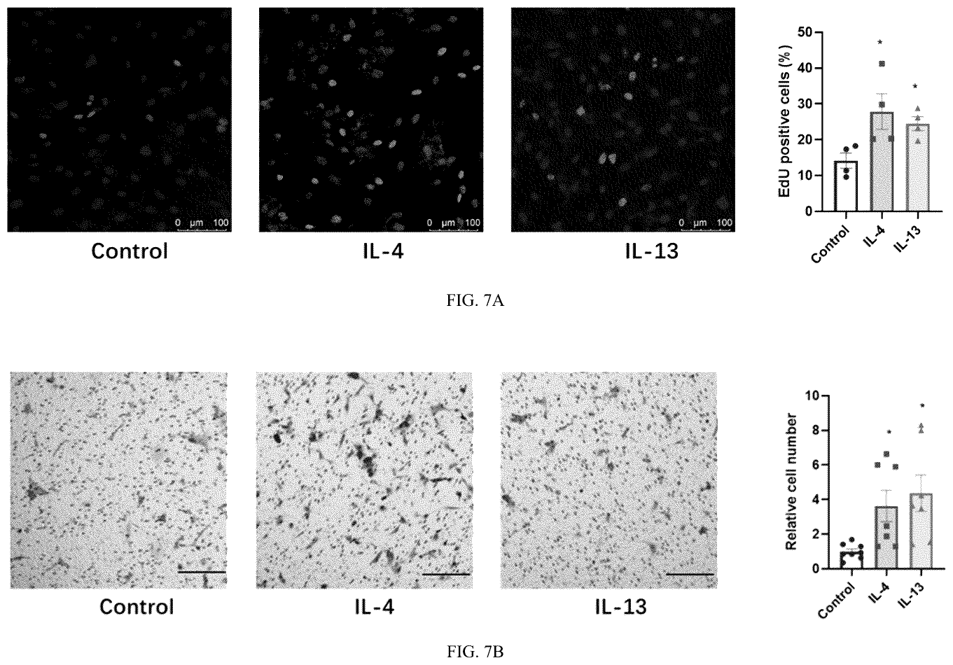

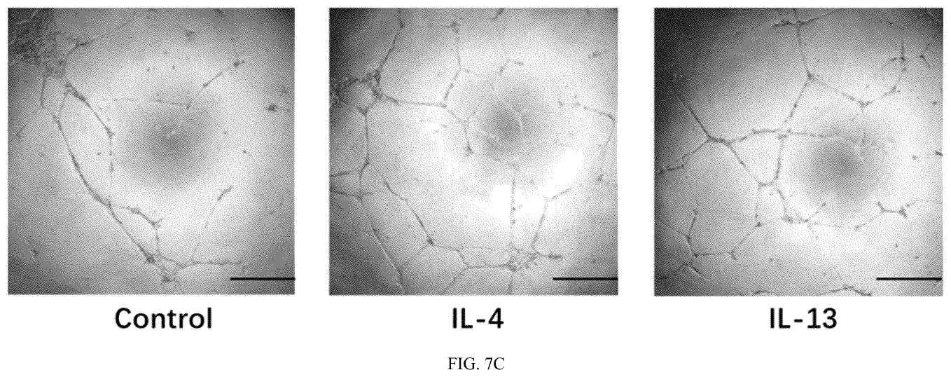

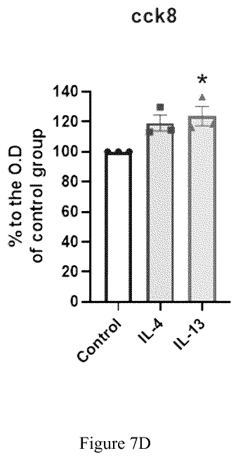

More importantly, IL-4/IL-13 treatment significantly promotes proliferation, migration and tube formation of endothelial cells. Notably, IL-4/IL-13 treatment significantly increases cell viability (FIGS. 7A-7D).

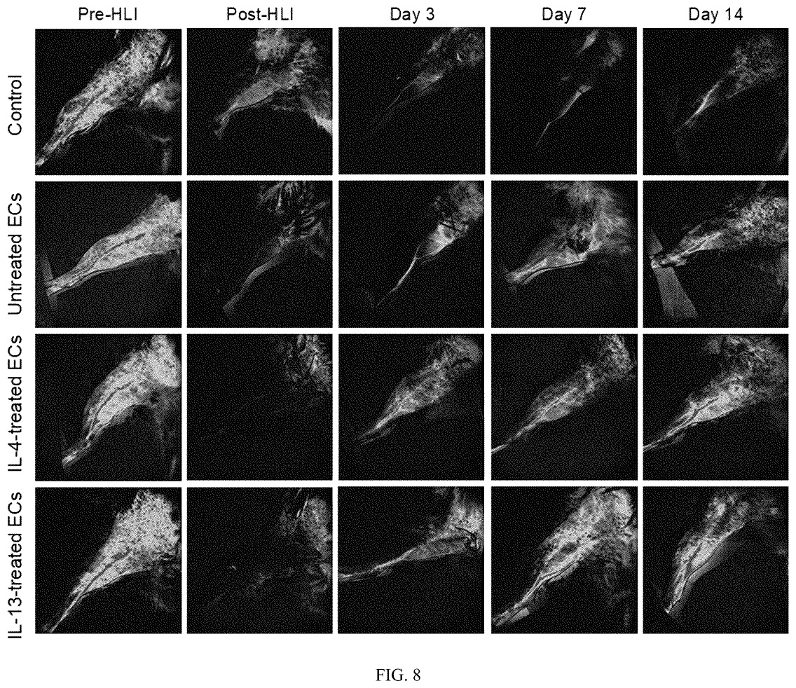

Treatment of IL-4 or IL-13 was administered to primary endothelial cells. Subsequently, the treated endothelial cells were injected into injured muscle. The blood flow of femoral arteries was significantly restored by injecting with IL-4-/IL-13-treated endothelial cells. Notably, the blood flow recovery of IL-4-/IL-13-treated endothelial cells injected groups was better than untreated endothelial cells injected groups (FIG. 8).

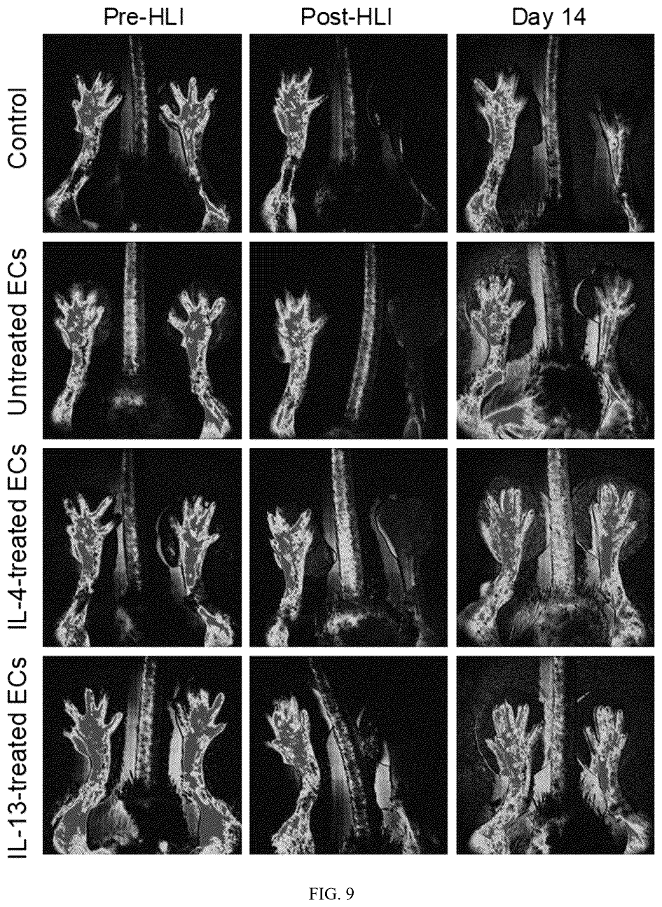

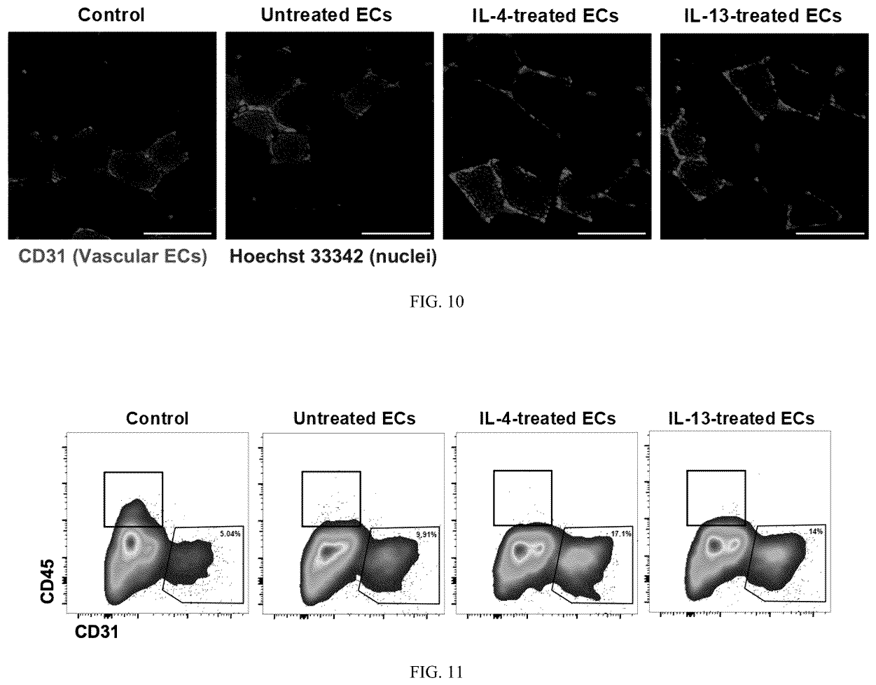

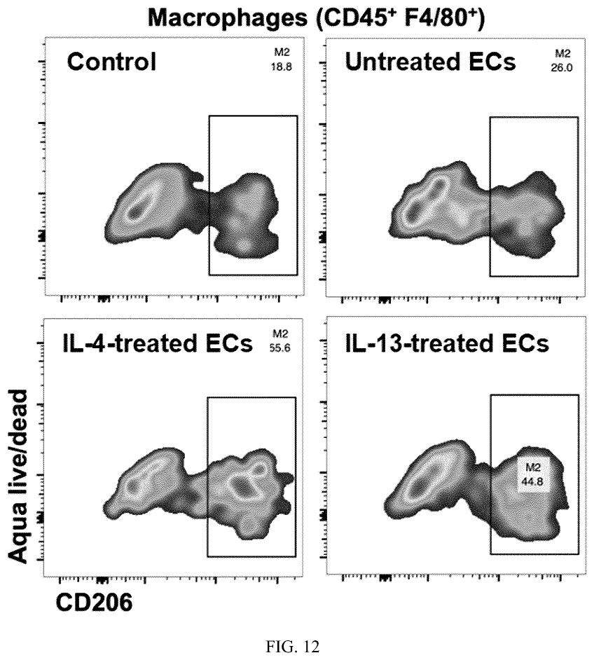

Besides, the blood flow of paws of IL-4-/IL-13-treated endothelial cells injected groups was remarkably restored, compared to the control group and untreated endothelial cells injected groups (FIG. 9).

The injection of IL-4-/IL-13-treated endothelial cells significantly promotes CD31 expression in injured mice muscle tissues, compared to control group and endothelial cells (untreated) injected groups (FIG. 10).

More impressively, the injection of IL-4-/IL-13-treated endothelial cells increased endothelial cells proportion in injured muscle tissues (FIG. 11).

Besides, the injection of IL-4-/IL-13-treated endothelial cells increases the M2 population in injured muscle tissues (FIG. 12).

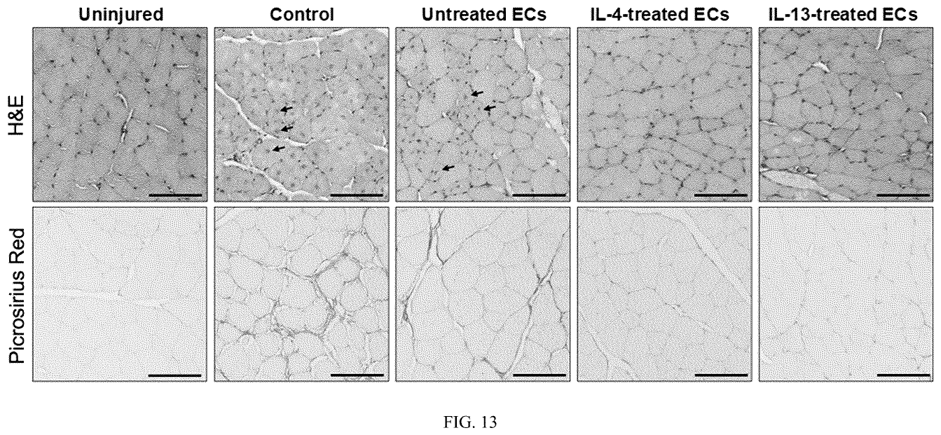

Compared to the control group and untreated endothelial cells injection groups, injection of IL-4-/IL-13-treated endothelial cells promoted muscle regeneration in injured muscle (FIG. 13).

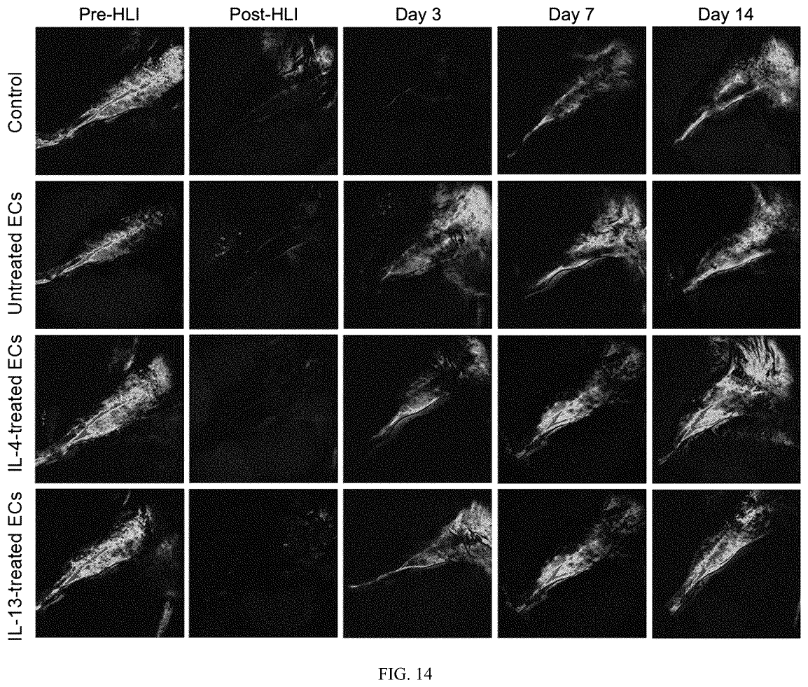

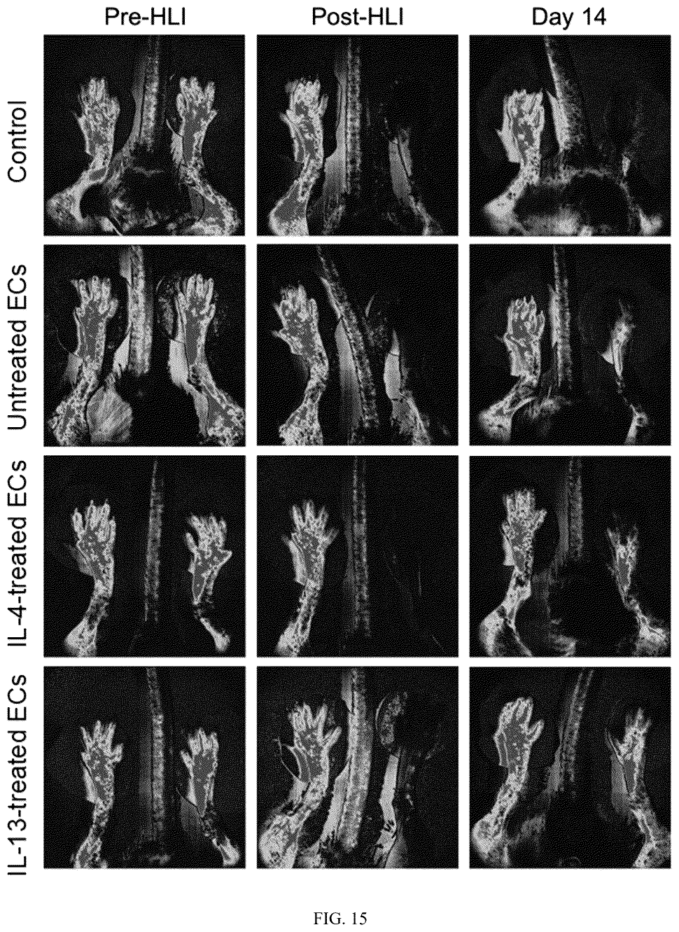

Impressively, similar effects of IL-4-/IL-13-treated endothelial cells on injured muscle were observed in diabetic mice. The injection of IL-4-/IL-13-treated endothelial cells significantly restored blood flow in injured muscle. More importantly, the blood flow recovery process of IL-4-/IL-13-treated endothelial cells injection group was better than endothelial cells (untreated) injection group (FIG. 14)

Besides, the blood flow of paws was restored by the injection of IL-4-/IL-13-treated endothelial cells, whose effects were better than control group and untreated endothelial cells group (FIG. 15).

### Example 2

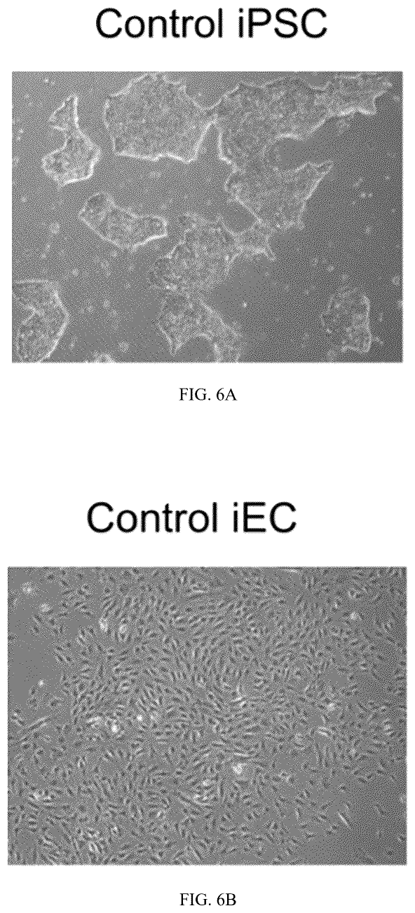

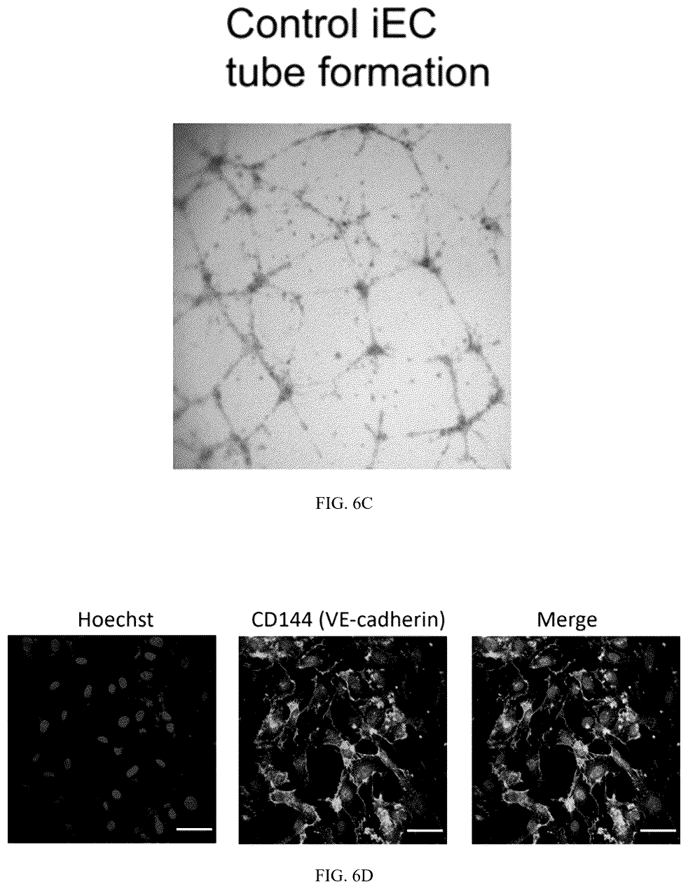

The subject invention specifically targets patients with advanced PAD, who (1) are not suitable for undergoing surgery; (2) show worsened progression with the use of medical treatments; and/or (3) show worsened progression while waiting for the surgery. The most potential group of patients are those with diabetes, smoking history and over 60 years old. The fibroblasts are first collected from a healthy patient in a sterile environment. The cells are grown in the Advanced Therapy Products (ATP) Good Manufacturing Practice (GMP) Centre. Once the cells are ready, the multiple sets of injecting the enhanced ECs intramuscularly can be performed by specialist out-patient services. An example showing functional endothelial cells derived from human fibroblasts that we generated is provided (FIGS. 6A-6D).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### Exemplary Embodiments

Embodiment 1. A method for treating peripheral arterial disease (PAD), the method comprising: (a) collecting fibroblasts and pluripotent stem cells (iPSCs) from a healthy subject; (b) obtaining induced endothelial cells (iECs) from the collected fibroblasts and induced pluripotent stem cell-derived endothelial cells (iPSC-ECs) from the iPSCs; (c) inducing the iPSC-ECs and iECs with IL-4/IL-13 to establish enhanced ECs; (d) administering an effective amount of the enhanced-endothelial cells to the PAD subject to promote angiogenesis and muscle tissue regeneration in ischemic muscle tissue.

Embodiment 2. The method of embodiment 1, wherein induction with IL-4/IL-13-treated endothelial cells (enhanced ECs) promotes muscle regeneration and revascularization in the PAD subject.

Embodiment 3. The method of any preceding embodiment, wherein treatment with IL-4 and IL-13 promotes a two to three fold increase in the number of induced endothelial cells derived from human iPSCs and fibroblasts when compared to the number of cells of an untreated group.

Embodiment 4. The method of any preceding embodiment, wherein treatment with IL-4 and IL-13 promotes about a three to four fold expression level increase of one or more of angiogenic genes IGF-1, VEGF-A, FGF-2, or GM-CSF in induced endothelial cells derived from human iPSCs and fibroblasts when compared to an untreated group.

Embodiment 5. The method of any preceding embodiment, wherein treatment with IL-4/IL-13-treated endothelial cells (enhanced ECs) promotes capillary tube assembly of endothelial cells in the ischemic muscle tissue, wherein tube length increase is about twofold, nodes increase is about three fold, and segment increase is about three fold when compared to untreated endothelial cells.

Embodiment 6. The method of any preceding embodiment, wherein treatment with IL-4/IL-13-treated endothelial cells (enhanced ECs) increases expression of CD31 in the ischemic muscle tissue by about three to four fold when compared to untreated endothelial cells.

Embodiment 7. The method of any preceding embodiment, wherein treatment with IL-4/IL-13-treated endothelial cells (enhanced ECs) increases an M2 macrophage population in the ischemic muscle tissue by at least about 1.7 to about 2.1 fold when compared to untreated endothelial cells.

Embodiment 8. The method of any preceding embodiment, wherein the treatment with IL-4/IL-13-treated endothelial cells (enhanced ECs) decreases the necrotic toe number by about 80%, decreases fibrosis within myofibers by about 50%, and promotes recovery and muscle tissue regeneration in the ischemic muscle tissue by about 100%.

Embodiment 9. The method of any preceding embodiment, wherein induction of IL-4/IL-13-treated endothelial cells (enhanced ECs) restores blood reperfusion by about 100%, promotes revascularization by about 50%, enhances capillary density by about 50%, and angiogenic program in ischemic muscle tissue by about 100%.

Embodiment 10. The method of any preceding embodiment, wherein the subject has a BMI over 30, has diabetes, and suffers from peripheral arterial disease.

Embodiment 11. The method of any preceding embodiment, wherein the collection of fibroblasts from the healthy subject is minimally invasive.

Embodiment 12. The method of any preceding embodiment, wherein the administering is done by intramuscular delivery.

Embodiment 13. The method of any preceding embodiment, wherein the administering is performed by autologous transplantation.

Embodiment 14. The method of any preceding embodiment, wherein the subject is a mammal.

Embodiment 15. The method of embodiment 14, wherein the mammal is a human.

Embodiment 16. The method of embodiment 15, wherein the human is a patient.

Embodiment 17. The method of embodiment 16, wherein the patient is healthy.

Embodiment 18. The method of embodiment 2, wherein induction with the enhanced ECs promotes about 100% muscle regeneration and about 50% revascularization in the PAD subject.

### REFERENCES

1. GBD 2019 Peripheral Artery Disease Collaborators. Global burden of peripheral artery disease and its risk factors, 1990-2019: a systematic analysis for the Global Burden of Disease Study 2019. Lancet Glob Health. 2023 October;11(10):e1553-e1565. doi: 10.1016/S2214-109X(23)00355-8

We claim:

1. A method for treating peripheral arterial disease (PAD), the method comprising: (a) collecting fibroblasts and pluripotent stem cells (iPSC) from a healthy subject; (b) obtaining induced endothelial cells (iECs) from the collected fibroblasts and induced pluripotent stem cell-derived endothelial cells (iPSC-ECs) from the iPSCs; (c) inducing the iPSC-ECs and iECs with IL-4/IL-13 to establish enhanced ECs; (d) administering an effective amount of the enhanced ECs to the PAD subject to promote angiogenesis and muscle tissue regeneration in ischemic muscle tissue.

2. The method of claim 1, wherein induction with IL-4/IL-13-treated endothelial cells (enhanced ECs) promotes muscle regeneration and revascularization in the PAD subject.

3. The method of claim 1, wherein treatment with IL-4 and IL-13 promotes a two to three fold increase in the number of induced endothelial cells derived from human iPSCs and fibroblasts when compared to the number of cells of an untreated group.

4. The method of claim 1, wherein treatment with IL-4 and IL-13 promotes about a three to four fold expression level increase of one or more of angiogenic genes IGF-1, VEGF-A, FGF-2, or GM-CSF in induced endothelial cells derived from human iPSCs and fibroblasts when compared to an untreated group.

5. The method of claim 1, wherein treatment with IL-4/IL-13-treated endothelial cells (enhanced ECs) promotes capillary tube assembly of endothelial cells in the ischemic muscle tissue, wherein tube length increase is about twofold, nodes increase is about three fold, and segment increase is about three fold when compared to untreated endothelial cells.

6. The method of claim 1, wherein treatment with IL-4/IL-13-treated endothelial cells (enhanced ECs) increases expression of CD31 in the ischemic muscle tissue by about three to four fold when compared to untreated endothelial cells.

7. The method of claim 1, wherein treatment with IL-4/IL-13-treated endothelial cells (enhanced ECs) increases M2 macrophage population in the ischemic muscle tissue by at least about 1.7 to about 2.1 fold when compared to untreated endothelial cells.

8. The method of claim 1, wherein the treatment with IL-4/IL-13-treated endothelial cells (enhanced ECs) decreases the necrotic toe number by about 80%, decreases fibrosis within myofibers by about 50%, and promotes recovery and muscle tissue regeneration in the ischemic muscle tissue by about 100%.

9. The method of claim 1, wherein induction of IL-4/IL-13-treated endothelial cells (enhanced ECs) restores blood reperfusion by about 100%, promotes revascularization by about 50%, enhances the capillary density by about 50%, and angiogenic program in ischemic muscle tissue by about 100%.

10. The method of claim 1, wherein the subject has a BMI over 30, has diabetes, and suffers from peripheral arterial disease.

11. The method of claim 1, wherein the collection of fibroblasts from the healthy subject is minimally invasive.

12. The method of claim 1, wherein the administering is done by intramuscular delivery.

13. The method of claim 1, wherein the administering is performed by autologous transplantation.

14. The method of claim 1, wherein the subject is a mammal.

15. The method of claim 14, wherein the mammal is a human.

16. The method of claim 15, wherein the human is a patient.

17. The method of claim 16, wherein the patient is healthy.

18. The method of claim 2, wherein induction with the enhanced ECs promotes about 100% muscle regeneration and about 50% revascularization in the PAD subject.
